(19) United States
(12) Patent Application Publication
(10) Pub. No.: US 2011/0160349 A1
(43) Pub. Date: Jun. 30, 2011

(54) WATER-REDUCED HYDRAULICALLY SETTING COMPOSITIONS WITH TEMPORALLY EXTENDED FLOW CAPABILITY

(75) Inventors: Lukas Frunz, Zurich (CH); Ueli Sulser, Unterengstringen (CH); Jorg Zimmermann, Zurich (CH); Jurgen Gresser, Laufenburg (DE)

(73) Assignee: SIKA TECHNOLOGY AG, Baar (CH)

(21) Appl. No.: 13/061,247

(22) PCT Filed: Aug. 26, 2009
(86) PCT No.: PCT/EP2009/060968

§ 371 Date: Feb. 28, 2011

(30) Foreign Application Priority Data
Aug. 26, 2008 (EP) 08162982.6

(51) Int. Cl. C04B 16/04; C07C 31/20; C08G 65/08; C04B 18/06; C04B 28/08; C04B 28/10; C04B 28/14

(52) U.S. Cl. 524/4; 568/852; 528/417; 525/409; 106/802; 106/708; 106/790; 106/795; 106/778

(57) ABSTRACT

A polymer P for prolonging the flowability of water-reduced aqueous hydraulically setting compositions, and a method for prolonging the flowability of water-reduced aqueous hydraulically setting compositions.

### TECHNICAL FIELD

The present invention relates to the field of additives for hydraulically setting systems, in particular dispersants for concrete compositions.

### PRIOR ART

Polymers composed of α,β-unsaturated carboxylic acids having polyalkylene glycol side chains, so-called polycarboxylates, have been used for quite some time in concrete technology as dispersants, in particular as superplasticizers, due to their intense water reduction. These polymers have a comb polymer structure. For the same water/cement (w/c) value, these polymers improve the workability of the concrete, or for the same workability, reduce the water demand and thus the w/c value, resulting in increased pressure-tightness and seal-tightness.

For the same workability, the conventional polycarboxylates reduce the water demand, and thus the w/c value, by approximately 20 to 30% compared to concrete compositions, which do not contain these polymers. Plasticizers having such an intense water reduction are referred to as “superplasticizers.” For certain applications, for example for ready-mix concrete of strength grade C20 having, for example, a cement proportion of 265 kg per cubic meter concrete and a water reduction rate of 10%, or ready-mix concrete of strength grade C30 having a cement proportion of 295 kg per cubic meter concrete and a water reduction rate of 12-15%, a fairly low water reduction rate of 15% maximum is desired. For conventional polycarboxylates, this may be achieved only by using same in a lower dosage, which results in a rapid decrease in flow table spread and therefore reduced workability. In particular for ready-mix concrete, however, prolonged workability is desired, since the concrete must be transported over a given period of time, for example from the production facility to the processing site. Accordingly, in the prior art, for applications having a low water reduction rate first-generation plasticizers, for example melamine-sulfonic acid-formaldehyde condensates, are used instead of superplasticizers. However, due to the release of toxic formaldehyde these plasticizers are problematic from an environmental standpoint, and therefore are not desirable. Other known plasticizers having a low water reduction rate are based on lignin sulfonates or naphthalene sulfonates, as described in WO 02/081400 A1, for example. Such plasticizers have the disadvantage that the compositions produced in this manner undergo discoloration. In addition, these known plasticizers must be used in relatively high dosages in order to achieve the desired water reduction rate while still ensuring greatly reduced workability. Furthermore, such first-generation plasticizers sometimes have inadequate workability despite a higher dosage.

It has been shown that the known concrete plasticizers may be used only to a limited extent for hydraulically setting compositions having a low water reduction rate while maintaining the same workability. The known concrete plasticizers must either be used in such small dosages that the workability is impaired, or they must be used in high dosages in order to achieve the desired water reduction rate, so that there is hardly any setting of the composition.

### DESCRIPTION OF THE INVENTION

The object of the present invention, therefore, is to provide dispersants for which the disadvantages of the prior art are overcome, and which are suitable for achieving a sufficient plasticizing effect, i.e., a desired water reduction rate of 5 to 15% and prolonged workability of hydraulically setting compositions.

Surprisingly, it has been found that this may be achieved using a polymer P according to claim 1. It has been determined that by using polymers containing at least one acid unit and at least one unit, preferably an ester and/or amide unit, which contains poly(oxyalkylene) groups, and having a molar ratio of the acid units to the units containing poly(oxyalkylene) groups which is between 1.2 and 1.7, the desired water reduction rate of 5 to 15% may be achieved with prolonged workability. It has also been shown that these polymers may be used for water reduction of hydraulically setting compositions, and that they result in neither excessive retardation nor acceleration of the setting. In addition, by using these polymers, compositions are possible which do not undergo discoloration.

The invention also encompasses a method for prolonging the flowability of water-reduced aqueous hydraulically setting compositions, and an aqueous hydraulically setting composition Z1. Further advantageous embodiments of the invention result from the subclaims.

### APPROACHES FOR CARRYING OUT THE INVENTION

The present invention relates to use of a polymer P for prolonging the flowability of water-reduced aqueous hydraulically setting compositions, wherein prolonging the flowability is characterized in that Z1 and Z2:
i.) have a flow table spread, directly after admixture of the water, of 220-180 mm according to EN 1015-3, or 450-550 mm according to EN 12350-5, and
ii.) have a difference in flow table spread directly after admixture of the water, compared to the flow table spread after 90 minutes, of 15% maximum according to EN 1015-3, or 20% maximum according to EN 12350-5.

Z1 refers to aqueous hydraulically setting compositions containing water and hydraulic binder, having a composition that is identical to aqueous hydraulically setting reference compositions Z2, except that Z1 also contains polymer P, and contains 5-15% less water than Z2.

The term “prolonging the flowability” is understood to mean that after admixture of a defined quantity of water and a given quantity of additives including polymer P, the hydraulically setting composition remains workable for a longer time than compositions, which contain no polymer P.

The flowability of aqueous hydraulically setting compositions is determined by one skilled in the art by means of the flow table spread, with knowledge of standards EN 1015-3 and EN 12350-5. In the present document, standard EN 12350-5 is used for determining the flow table spread of hydraulically setting compositions containing additives [with a particle size] greater than 8 mm.

A flow table spread, directly after admixture of the water, of 220-180 mm according to EN 1015-3, or 450-550 mm according to EN 12350-5, is advantageous in that for values above 220 mm for an aqueous hydraulically setting composition, the initial flow table spread is too high, and the composition may separate and therefore may no longer be usable.

A difference in flow table spread directly after admixture of the water, compared to the flow table spread after 90 minutes, of 15% maximum according to EN 1015-3, or 20% maximum according to EN 12350-5, has the advantage of advantageous flowability over the entire period of 90 minutes after admixture of the water.

The workability, i.e., the flow table spread, of Z1 as the result of using polymer P at the start, i.e., directly after admixture of the mixing water with the hydraulic binder, as well as after 90 min after admixture of the mixing water, is therefore similar to that of reference composition Z2.

When reference composition Z2 is a hydraulically setting composition which has an advantageous flow table spread directly after admixture of the mixing water with the hydraulic binder, and whose flow table spread after 90 minutes is more or less maintained, or decreases only slightly, depending on the composition, a maximum of 15% or 20%, use according to the invention of polymer P results in comparable properties of Z1, with the advantage that Z1 contains 5-15% less water than Z2. This results in increased pressure-tightness and seal-tightness in the set state for Z1 compared to Z2.

As the result of using polymer P, the workability, i.e., the flow table spread, of Z1 is therefore similar to that of reference composition Z2 at the start, i.e., directly after admixture of the mixing water with the hydraulic binder, as well as after 90 min.

When reference composition Z2 is a hydraulically setting composition which has an advantageous flow table spread directly after admixture of the mixing water with the hydraulic binder, and whose flow table spread after 90 minutes is more or less maintained, or decreases only slightly, depending on the composition, preferably less than 15 to 20%, use according to the invention of polymer P results in comparable properties of Z1, with the advantage that Z1 contains 5-15% less water than Z2. This results in increased pressure-tightness and seal-tightness in the set state for Z1 compared to Z2. In addition, by using polymer P, compositions are possible which do not undergo discoloration.

This is a major advantage compared to the use of conventional plasticizers. When conventional superplasticizers are used, the dosages used must be small enough to prevent excessively high flow table spread directly after admixture of the mixing water, which would cause the composition to separate because the flow table spread would be too low after 90 minutes. When conventional first-generation plasticizers, for example the lignosulfonates, are used, much higher dosages compared to polymer P must be used, which has major environmental as well as economic disadvantages.

The term “hydraulically setting composition” basically may be understood to mean all hydraulically setting substances known to one skilled in the art in the field of concrete. In the present case, this involves in particular hydraulic binders such as cements, for example Portland cements or alumina cements and the respective mixtures thereof with fly ash, fumed silica, slag, granulated blast furnace slag, and limestone filler. Further hydraulically setting substances within the meaning of the present invention are gypsum in the form of the anhydrite or hemihydrate, or quicklime. Cement is preferred as hydraulically setting composition. In addition, additives such as sand, gravel, rock, quartz powder, chalk, and common components such as other concrete plasticizers, for example lignosulfonates, sulfonated naphthalene-formaldehyde condensates, sulfonated melamine-formaldehyde condensates, or polycarboxylate ethers, accelerators, corrosion inhibitors, retardants, shrinkage reducing agents, antifoaming agents, or pore formers are possible as additives.

The hydraulic binder is preferably selected from the group comprising cement; mixtures of cement with fly ash, fumed silica, slag, granulated blast furnace slag, or limestone filler; gypsum; and quicklime. Cement is particularly preferred.

Polymer P includes:
a) m mol-% of at least one acid unit A of formula (I);

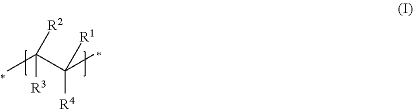

and
b) n mol-% of at least one structural unit B of formula (II);

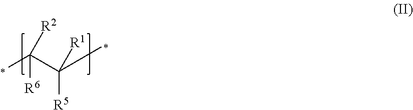

and optionally
c) p mol-% of at least one further structural unit C.

R¹ and R² independently stand for H, COOM, CH₂COOM, or an alkyl group containing 1 to 5 carbon atoms, in particular for H or CH₃; R³ independently stands for H, CH₃, COOM, or CH₂COOM, in particular for H; and R⁴ independently stands for a radical of carboxylic acid, in particular for COOM; or R³ together with R⁴ may form a ring to give —CO—O—CO—.

M stands for H, alkali metal, alkaline earth metal, or other bivalent or trivalent metal atoms, ammonium, alkylammonium, or a mixture thereof. M may in particular represent H, Na, Ca/2, Mg/2, NH₄, or an organic ammonium. It is clear to one skilled in the art that for the polyvalent ions an additional counterion must be present which, among others, may be a carboxylate itself or another molecule of polymer P. The ammonium compounds are in particular tetraalkylammonium or also HR³N, where R represents an alkyl group, in particular a C₁ to C₆ alkyl group, preferably ethyl or butyl. Ammonium ions are obtained in particular by neutralization of the carboxyl group with commercially available tertiary amines.

R⁵ independently stands for a radical of formula (III)

—(CH₂)ₓ—R⁷—(R⁸O)_{y}—R⁹ (III)

In this regard, R¹ independently stands for an ester, ether, amide, or imide connecting element, preferably for an ester or amide connecting element, in particular for —COO— or —CO—NH—. R⁸ stands for a C₂-C₆ alkylene group, preferably a C₂-C₄ alkylene group, or a mixture of C₂, C₃, and/or C₄ alkylene groups in any given sequence; and R⁹ stands for H, a C₁-C₁₂ alkyl or cycloalkyl radical, a C₇-C₂₀ alkylaryl or aralkyl radical, or a substituted or unsubstituted aryl radical, or a monofunctional organic radical containing 1 to 30 C atoms and optionally containing heteroatoms.

R⁶ independently stands for H, CH₃, COOM, or CH₂COOM, or a substituent such as defined for R⁵, preferably H.

The subscript x independently has the value 0 or 1; and y independently stands for the value 3-250, preferably 5-120.

m, n, and p independently stand for numbers, wherein the sum m+n+p=100, and m>0, n>0, and p≧0; and the ratio m/n is between 1.2 and 1.7.

It is preferred that m stands for a number from 30 to 66, preferably from 50 to 63, n stands for a number from 20 to 50, preferably from 34 to 44, and p stands for a number from 0 to 40, preferably from 0 to 1. The ratio m/n means the molar ratio of all carboxylic acid units A to all structural units B, i.e., to all units in polymer P which include poly(oxyalkylene) groups. Particularly good results are obtained when this ratio is between 1.2 and 1.6.

Examples of suitable acid units A are units which result from polymerization of acrylic acid, methacrylic acid, mesaconic acid, citraconic acid, glutaconic acid, fumaric acid, maleic acid, maleamic acid, itaconic acid, vinylbenzoic acid, crotonic acid, or derivatives or analogs thereof. Monocarboxylic acids are preferred. Particularly suited as acid unit A is a unit which results from polymerization of a (meth)acrylic acid unit or a salt thereof. In the entire present document, “(meth)acrylic acid” is understood to mean acrylic acid as well as methacrylic acid, or mixtures thereof.

The at least one acid unit A of formula (I) is preferably partially or completely neutralized. The acid unit may be present as a free acid or also as a salt or partial salt, wherein the term “salt” here and below includes, in addition to the classical salts such as those obtained by neutralization with a base, chemically complexed compounds between metal ions and the carboxylate or carboxyl groups as ligands.

In one preferred embodiment, in polymer P R⁵ independently stands for —COO—(R⁸O)_{y}—R⁹ and/or —CO—NH—(R⁸O)_{y}—R⁹, in particular for —COO—(R⁸O)_{y}—R⁹, or a mixture of COO—(R⁸O)_{y}—R⁹ and —CO—NH—(R⁸O)_{y}—R⁹, and —(R⁵O)_{y}— stands for a C₂-C₄ polyoxyalkylene group, for example a polyoxyethylene group, a polyoxypropylene group, a polyoxybutylene group, or a polymisobutylene group, in particular for a polyoxyethylene group or a polyoxypropylene group, or mixtures of oxyethylene and oxypropylene units in any possible sequence, for example random, alternating, or block, and y stands for 3 to 250, preferably 10 to 120. In one preferred polymer P, at least 30 mol-%, particularly preferably 50-100 mol-%, more preferably 80-100 mol-%, most preferably 100 mol-%, of structural unit B of formula (II) is represented by a structure in which R⁸ stands for a C₂ alkylene group. That is, R⁵ preferably contains at least 30 mol-% (C₂H₄O) units, preferably 50 to 100 mol-% (C₂H₄O) units, more preferably 80 to 100 mol-% (C₂H₄O) units, relative to the total molar quantity of all (R⁸O) units. In particular, R⁵ preferably contains 100 mol-% (C₂H₄O) units relative to the total molar quantity of all (R⁸O) units. Depending on the preparation method for polymer P, R⁹ may stand for H, a C₁-C₁₂ alkyl or cycloalkyl radical, a C₇-C₂₀ alkylaryl or aralkyl radical, or a substituted or unsubstituted aryl radical, or a monofunctional organic radical containing 1 to 30 C atoms and optionally containing heteroatoms. If polymer P is prepared via the polymer-analogous reaction, R⁹ is preferably a methyl radical, and does not stand for a hydrogen atom.

A polymer P is particularly preferred in which R¹ is H or CH_{B}, and R², R³, and R⁶ stand for H, and R⁴ is COOM, and M is H or an alkali or alkaline earth metal. The acid unit A of formula (I) thus preferably represents an acrylic or methacrylic acid unit or salts thereof.

Further structural unit C may include a further ether, ester, amide, or imide unit, preferably an amide or ester unit. For example, further structural unit C may contain esters of carboxylic acid, sulfonic acid, phosphoric acid, phosphonic acid, or carbonylamidomethylpropanesulfonic acid, and the alkali or earth alkaline salts thereof, poly(oxyalkylene)oxycarbonyl, poly(oxyalkylene)aminocarbonyl, poly(oxyalkylene)oxyalkyl, poly(oxyalkylene)oxy, hydroxyethyloxycarbonyl, acetoxy, phenyl, or N-pyrrolidonyl groups. Further structural unit C preferably contains polyoxyalkylene groups, preferably polyoxyethylene groups, polyoxypropylene groups, or mixtures thereof. For example, structural unit C may be an ester unit which is prepared by reacting a mono- or dicarboxylic acid with an alkyl alcohol, in particular a C₆-C₂₀ alkyl alcohol.

One particularly preferred polymer P contains or comprises

a) m mol-% of at least one acid unit A of formula (I′):

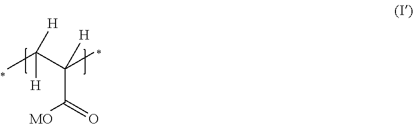

and

b) n mol-% of at least one structural unit B of formula (In;

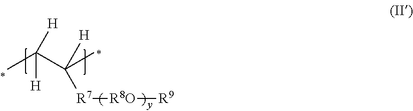

where M represents H, Na, Ca/2, Mg/2, NH₄, or an organic ammonium, preferably H,

R⁷ stands for COO or CONH,

R⁸ stands for an ethylene group,

R⁹ stands for a C₁ to C₁₂ alkyl group, preferably a methyl group,

y stands for 3-250, preferably 10-100,

and the molar ratio m/n is between 1 and 2, in particular between 1.2 and 1.6.

Polymer P may contain a combination of various structural units of the respective structural units A, B, and C. For example, multiple structural units A may be present in mixed form in polymer P, such as a mixture of methacrylic acid units with acrylic acid units, for example. Or, multiple different ester and/or amide units B may be present in mixed form in polymer P, such as multiple ester units B having various substituents R⁸, for example. Preferred, for example, is the joint use of poly(oxyalkylenes), in particular poly(oxyethylene) with poly(oxypropylene), or the joint use of poly(oxyalkylenes), in particular poly(oxyethylene), having different molecular weights.

In one preferred embodiment, polymer P contains 30 to 66 mol-%, preferably 50 to 63 mol-%, of acid unit A of formula (I), 20 to 50 mol-%, preferably 34 to 44 mol-%, of structural unit B of formula (II), and optionally 0 to 40 mol-% of structural unit C, in each case relative to the total molar quantity of structural units A, B, and C in polymer P.

The sequence of the individual structural units A, B, and C in polymer P may be alternating, statistical, block, or random.

Polymer P preferably has a molecular weight M_{w} in the range of 10,000-150,000 g/mol, preferably 15,000-100,000 g/mol, particularly preferably 20,000-80,000 g/mol.

Within the meaning of the invention, “molecular weight” or “molar weight” refers to the average molecular weight M_{w}.

Polymer P may be prepared in various ways. Essentially two methods are in use. In a first method, the polymers are prepared from a polycarboxylate and the respective alcohols and/or amines in a so-called polymer-analogous reaction. In a second method, the polymers are prepared from the respective unsaturated carboxylic acid-, and ester-, ether-, amide-, and/or imide-functional monomers via radical polymerization.

Particularly preferred polymers are prepared in the polymer-analogous reaction according to the first method. The polymer-analogous reaction has the major advantage that, using commercially available polymers of α,β-unsaturated acids, in particular mono- or dicarboxylic acids, in particular poly(meth)acrylic acids, a great variety of comb polymers having very different properties may be easily and reliably obtained by varying the quantity, type, and ratio of alcohol and amine. Such polymer-analogous reactions are described in WO 97/35814 A1, WO 95/09821 A2, DE 100 15 135 A1, EP 1 138 697 A1, EP 1 348 729 A1, and WO 2005/090416 A1, for example. Details concerning the polymer-analogous reaction are disclosed, for example, in EP 1 138 697 B1 on page 7, line 20 to page 8, line 50 and in the examples thereof, or in EP 1 061 089 B1 on page 4, line 54 to page 5, line 38 and in the examples thereof. Polymer P may also be obtained in the solid aggregate state, as described in EP 1 348 729 A1 on pages 3-5 and in the examples thereof.

Thus, a polymer P is preferably used in which polymer P is obtainable via the reaction of (a) at least one polycarboxylic acid or an analog of a polycarboxylic acid; and (b) at least one monohydroxy compound E and/or at least one monoamine compound F containing at least one polyoxyalkylene group, and optionally (c) at least one further compound D.

“Polycarboxylic acid or an analog of a polycarboxylic acid” refers to a home- or copolymer which may be obtained by polymerization of at least one monomer a and optionally at least one monomer b. Monomer a is selected from the group comprising unsaturated monocarboxylic acids, unsaturated dicarboxylic acids, and analogs thereof and mixtures thereof. Unsaturated mono- or dicarboxylic acids preferably comprise maleic acid, itaconic acid, fumaric acid, citraconic acid, glutaconic acid, mesaconic acid, or crotonic acid, in particular acrylic acid or methacrylic acid. Within the meaning of the present invention, “analog of a mono- or dicarboxylic acid or polycarboxylic acid” refers to acid salts, acid halides, acid anhydrides, and acid esters, in particular alkyl acid esters.

Monomer b is preferably selected from the group of ethylenically unsaturated monomers containing α,β-unsaturated mono- or dicarboxylic acids, α,β-unsaturated mono- or dicarboxylic acid esters, α,β-unsaturated carboxylates, styrene, ethylene, propylene, vinyl acetate, in particular methacrylic acid, acrylic acid, crotonic acid, itaconic acid, maleic acid, fumaric acid, and the salts and esters thereof, and mixtures thereof.

A copolymer of acrylic acid and methacrylic acid and the salts or partial salts thereof is preferred as copolymer.

Polymethacrylic acid or the salts or partial salts thereof is preferred as homopolymer.

The polycarboxylic acid or the analog of the polycarboxylic acid may be present as a free acid or as a partial salt, wherein the term “salt” here and below includes, in addition to the classical salts such as those obtained by neutralization with a base, chemically complexed compounds between metal ions and the carboxylate or carboxyl groups as ligands. In the preparation of polycarboxylic acid or the analog of polycarboxylic acid, any initiators, co-initiators, and polymerization regulators used are optionally selected in such a way that preferably no reactive hydroxyl or amine functions are present in polymer P.

Here and below, “monohydroxy compound” refers to a substance which contains only one free hydroxyl group.

Here and below, “monoamine compound” refers to a substance which contains only one free amino group.

The home- or copolymer of polycarboxylic acid or of the analog of polycarboxylic acid is obtained via radical polymerization according to customary methods, in solvent, preferably in water or in a substance. This radical polymerization is preferably carried out in the presence of at least one molecular weight regulator, in particular an inorganic or organic sulfur compound, for example mercaptans, or a phosphorus compound. The home- or copolymer of polycarboxylic acid or of the analog of polycarboxylic acid preferably has a molecular weight M_{w} of 500 to 20,000 g/mol, preferably 2000 to 10,000 g/mol, particularly preferably 3500 to 6500 g/mol.

The monohydroxy compound E is preferably terminated at one end by end groups which are nonreactive under customary reaction conditions. This is preferably a polymer having a polyalkylene glycol base structure. The monohydroxy compound E has the formula (IV)

HO—(R⁸O)_{y}—Fe (IV),

where R⁸ independently stands for a C₂-C₄ alkylene group having any possible sequence of the (R⁸O) units; R^{9′} stands for a C₁-C₁₂ alkyl or cycloalkyl radical, a C₇-C₂₀ alkylaryl or aralkyl radical, or a substituted or unsubstituted aryl radical, or a monofunctional organic radical containing 1 to 30 C atoms and optionally containing heteroatoms; and y independently stands for 3 to 250, preferably 10 to 120.

Monohydroxy compounds E of formula (IV) containing a methyl, ethyl, isopropyl, or n-butyl group, in particular a methyl group, are preferred as substituent R⁹. R⁸ preferably independently stands for a C₂ alkylene group and/or a C₃ alkylene group. E is preferably a mixed polymer of ethylene oxide/propylene oxide, more preferably polyethylene glycol terminated at one end by end groups.

Mixtures of multiple different compounds of group E are also possible. Thus, for example, polyethylene glycols of different molecular weights which are terminated at one end by end groups may be mixed, or, for example, mixtures of polyethylene glycols terminated at one end by end groups with mixed polymers of ethylene oxide and propylene oxide terminated at one end by end groups or polypropylene glycols terminated at one end by end groups may be used.

Within the meaning of the invention, “terminated by end groups which are nonreactive under customary reaction conditions” is understood to mean that, instead of functional groups which are reactive for esterification or amidation, groups are present which are nonreactive. The customary reaction conditions are those familiar to one skilled in the art for esterifications and amidations. In compounds “terminated at one end,” only one end is nonreactive.

In one preferred embodiment, the monohydroxy compound E is a polyalkylene glycol, terminated at one end by end groups, having a molecular weight M_{w} of 500 to 10,000 g/mol, in particular 800 to 8000 g/mol, preferably 1000 to 6000 g/mol. Also suitable is a mixture of polyalkylene glycols of different molecular weights terminated at one end by end groups, for example the mixture of polyalkylene glycols having a molecular weight of 1000 g/mol with polyalkylene glycols having a molecular weight of 5000 g/mol.

In the first method, a monoamine compound F may be used in addition to monohydroxy compound E or instead of monohydroxy compound E. Amide groups are formed in this manner. Typical examples of such monoamine compounds F may be represented by formula (V):

NH₂—(R⁸O)_{y}—R⁹ (V)

Substituents R⁸ and R⁹ and subscript y independently have the same meanings as previously defined for formula (III). Depending on the preparation method for polymer P, R⁹ in formula (V) may stand for H, a C₁-C₁₂ alkyl or cycloalkyl radical, a C₇-C₂₀ alkylaryl or aralkyl radical, or a substituted or unsubstituted aryl radical, or a monofunctional organic radical containing 1 to 30 C atoms and optionally containing heteroatoms. If polymer P is prepared via the polymer-analogous reaction, and if the optionally formed anhydride groups are not completely or partially reacted with an amine compound to produce an amide in a second step, R⁹ of formula (V) is preferably R^{9′}, in particular a methyl radical, and does not stand for a hydrogen atom.

Examples of such monoamine compounds F are α-methoxy-ω-aminopolyoxyethylene, α-methoxy-ω-aminopolyoxypropylene, and α-methoxy-ω-amino-oxyethylene-oxypropylene copolymer.

Particularly preferred as monoamine compounds F are α-methoxy-ω-amino-oxyethylene-oxypropylene copolymers, for example JEFFAMINE M-2070, or α-methoxy-ω-aminopolyoxyethylenes, as well as other monoamines marketed, for example, by Huntsman under the name JEFFAMINE of the M series, and mixtures thereof. α-Methoxy-ω-amino-oxyethylene-oxypropylene copolymers are most preferred. Such monoamine compounds F are obtainable, for example, from a polymerization of ethylene oxide and/or propylene oxide initiated by alcohol, followed by conversion of the terminal alcohol group to an amine group.

As further compound D, a compound is preferred which is able to react with polycarboxylic acid or the analog of polycarboxylic acid. Examples of a compound D include further amines or alcohols, for example a C₆-C₂₀ alkyl alcohol or a further mono- or diamine. Multiple different compounds D may also be used.

The reaction of polycarboxylic acid or the analog of polycarboxylic acid with at least one monohydroxy compound E and/or with at least one monoamine compound F, and optionally a compound D, to produce a polymer P is typically carried out in the polymer-analogous reaction in such a way that the at least one monohydroxy compound E and/or the at least one monoamine compound F is/are added to the polycarboxylic acid or the analog of polycarboxylic acid with stirring, and heated to the reaction temperature. With continued stirring the mixture is reacted, possibly under vacuum or by passing a gas stream over or through the reaction mixture. The temperature for this reaction is 140° to 200° C., for example. However, the reaction is also possible at temperatures of 150° C. to 175° C. If a monoamine compound F is used in addition to the monohydroxy compound E, it may be added at the same time as the monohydroxy compound E, or at a later time during this reaction step.

In one preferred embodiment, this reaction is carried out in the presence of an esterification catalyst, in particular an acid. The acid is preferably sulfuric acid, p-toluenesulfonic acid, benzenesulfonic acid, methanesulfonic acid, phosphoric acid, or phosphorous acid. Sulfuric acid is preferred. The water may be removed from the reaction mixture under atmospheric pressure or under vacuum. In addition, a gas stream may be passed over or through the reaction mixture. Air or nitrogen may be used as the gas stream.

The reaction may be monitored by measuring the acid number, for example by titration, and terminated at a desired acid number so that the desired acid content is achieved. The reaction is terminated by discontinuing the vacuum and cooling.

In one preferred embodiment, a polyacrylic acid containing a polyoxyethylene that is terminated at one end by a methyl group is esterified and/or reacted with a monoamine.

In the so-called polymer-analogous reaction, in addition to ester groups and optionally amide groups, anhydride groups may also be formed, which in a second step may be completely or partially reacted with an amine compound to form an amide. Such methods are described in WO 2005/090418 A1, for example.

In a second preparation method, polymer P is prepared via radical polymerization. The path via radical polymerization is the most common method; however, for specialized compounds it is made more difficult by the commercially availability of the corresponding monomers, and requires complicated process control.

Thus, it may be advantageous when polymer P is obtainable by the polymerization reaction, in the presence of at least one radical former, of

(a) at least one ethylenically unsaturated monomer M which is selected from the group comprising unsaturated mono- or dicarboxylic acids, unsaturated sulfonic acids, unsaturated phosphoric acids, unsaturated phosphonic acids, or the salts thereof; with

(b) at least one ethylenically unsaturated carboxylic acid derivative H of formula (VI);

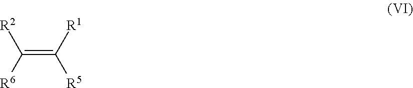

and optionally

(c) at least one further ethylenically unsaturated compound L.

Substituents R², R⁵, and R⁶ each independently have the same meanings as described for formula (II).

The ethylenically unsaturated monomer M is preferably a mono- or dicarboxylic acid or the salt of the unsaturated mono- or dicarboxylic acid. The mono- or dicarboxylic acid is preferably acrylic acid or methacrylic acid, maleic acid, itaconic acid, fumaric acid, citraconic acid, glutaconic acid, mesaconic acid, or crotonic acid, in particular acrylic acid or methacrylic acid. Acrylic acid is particularly preferred.

The at least one ethylenically unsaturated carboxylic acid derivative H of formula (VI) is preferably a carboxylate or carboxamide, particularly preferably an acrylate or a methacrylate. Poly(oxyalkylene) acrylates represent preferred examples of such esters. Multiple monomers of formula (VI) having various substituents R⁵ may be used in combination with one another. Preferred, for example, is the joint use of various poly(oxyalkylenes), in particular poly(oxyethylene) with poly(oxypropylene), or the joint use of poly(oxyethylenes) having different molecular weights for the poly(oxylakylene) (meth)acrylates or (meth)acrylamides. Possible examples of suitable carboxamides are amides of ethylenically unsaturated mono- or dicarboxylic acids with amine compounds. Amides of (meth)acrylic acid, preferably the poly(oxyalkylene) monoamides, are particularly preferred. Particularly preferred amide monomers are thealkylpoly(oxyalkylene) (meth)acrylamides, particularly preferably the methyl poly(oxyethylene) (meth)acrylamides, the methylpoly(oxyethylene)poly(oxypropylene) (meth)acrylamides, or the methyl(polyoxypropylene) (meth)acrylamides. One or more of these unsaturated carboxamides may be used.

The further ethylenically unsaturated compound L is preferably a carboxylate or carboxamide, particularly preferably an acrylate or acrylamide, or a methacrylate or methacrylamide. Examples of such esters or amides are poly(oxyalkylene) (meth)acrylates or poly(oxyalkylene) (meth)acrylamides. Multiple different compounds L may be used in combination with one another. Mono- or dihydroxyethyl (meth)acrylamide or mono- or dihydroxypropyl (meth)acrylamide, mono- or dicyclohexyl (meth)acrylamide, N-alkyl or N-hydroxyethyl (meth)acrylamide, or N-alkyl or N-hydroxypropyl (meth)acrylamide, for example, are suitable.

If polymer P is used in liquid form, a solvent is preferably used for the reaction. Examples of preferred solvents include alcohols, in particular ethanol or isopropanol, and water, with water being the most preferred solvent.

Polymer P may also be present in the solid aggregate state. Within the meaning of the invention, “polymers in the solid aggregate state” are understood to mean polymers which are in the solid aggregate state at room temperature, and are powders, flakes, pellets, granules, or plates, for example, and which may be easily transported and stored in this form.

In the solid aggregate state, polymer P may be a component of a so-called dry mixture, for example a cement composition, which is storable over extended periods and is typically packed in bags or stored in silos and used. Such a dry mixture may also be used after extended periods of storage, and has good pourability.

Polymer P may also be added to a customary hydraulically setting composition together with or shortly before or shortly after the admixture of the water. It has proven to be particularly suitable to add the polymer P in the form of an aqueous solution or dispersion, in particular as mixing water or as part of the mixing water. The aqueous solution or dispersion is prepared by adding water in the production of polymer P, or by subsequently mixing polymer P with water. A dispersion or a solution is obtained, depending on the type of polymer P. A solution is preferred.

It may also be advantageous for the aqueous hydraulically setting composition Z1 to additionally contain a plasticizing agent selected from the group comprising lignosulfonate, naphthalenesulfonic acid-formaldehyde condensate, sulfonated melamine-formaldehyde condensate, molasses, and gluconate.

In a further aspect, the present invention relates to a method for prolonging the flowability of water-reduced aqueous hydraulically setting compositions, wherein polymer P is also added to an aqueous hydraulically setting composition Z1 containing water and hydraulic binder, which except for a 5-15% lower water content has a composition identical to an aqueous hydraulically setting reference composition Z2. Z1 and Z2:
i.) have a flow table spread, directly after admixture of the water, of 220-180 mm according to EN 1015-3, or 450-550 mm according to EN 12350-5, and
ii.) have a difference in flow table spread directly after admixture of the water, compared to the flow table spread after 90 minutes, of 15% maximum according to EN 1015-3, or 20% maximum according to EN 12350-5.

Polymer P is a polymer P as described above. Polymer P may be used in liquid as well as solid form.

The hydraulically setting composition is the same as that previously mentioned. The hydraulic binder is preferably selected from the group comprising cement; mixtures of cement with fly ash, fumed silica, slag, granulated blast furnace slag, or limestone filler; gypsum; and quicklime. Cement is particularly preferred.

It may also be advantageous for the aqueous hydraulically setting compositions Z1 to additionally contain a plasticizing agent selected from the group comprising lignosulfonate, naphthalenesulfonic acid-formaldehyde condensate, sulfonated melamine-formaldehyde condensate, molasses, and gluconate.

The present invention further relates to an aqueous hydraulically setting composition Z1 containing water and hydraulic binder, having a composition that is identical to an aqueous hydraulically setting reference composition Z2, except that Z1 also contains polymer P, and contains 5-15% less water than Z2. Z1 and Z2:
i) have a flow table spread, directly after admixture of the water, of 220-180 mm according to EN 1015-3, or 450-550 mm according to EN 12350-5, and
ii.) have a difference in flow table spread directly after admixture of the water, compared to the flow table spread after 90 minutes, of 15% maximum according to EN 1015-3, or 20% maximum according to EN 12350-5.

Polymer P is a polymer P as described above.

The hydraulically setting composition Z1 is the same as that previously mentioned. The hydraulic binder is preferably selected from the group comprising cement; mixtures of cement with fly ash, fumed silica, slag, granulated blast furnace slag, or limestone filler; gypsum; and quicklime. Cement is particularly preferred.

It may also be advantageous for Z1 to additionally contain at least one plasticizing agent selected from the group comprising lignosulfonate, naphthalenesulfonic acid-formaldehyde condensate, sulfonated melamine-formaldehyde condensate, molasses, and gluconate.

### EXAMPLES

The invention is explained in greater detail with reference to examples.

### 1. Polymers P Used

TABLE 1

| Abbrevia- | | |
|---|---|---|
| tion | Meaning | Mw |
| PEG520 | Poly(oxyethylene) without terminal OH groups | 520 g/mol |
| PEG1000 | Poly(oxyethylene) without terminal OH groups | 1000 g/mol |
| PEG3000 | Poly(oxyethylene) without terminal OH groups | 3000 g/mol |
| PEG5000 | Poly(oxyethylene) without terminal OH groups | 5000 g/mol |
| EO/PO | Block copolymer of ethylene oxide and | 2000 g/mol |
| (70/30) | propylene oxide in a 70:30 ratio, without | |
| 2000 | terminal OH groups | |

Abbreviations used.
Mw = average molecular weight

Polymers P-1, P-2, and P-4 through P-8 as well as comparative examples V-1 through V-3 listed in Table 2 were prepared in a known manner by polymer-analogous reaction of polyacrylic acid with the corresponding alcohols and/or amines. Details concerning the polymer-analogous reaction are disclosed, for example, in EP 1 138 697 B1 on page 7, line 20 to page 8, line 50 and in the examples thereof, or in EP 1 061 089 B1 on page 4, line 54 to page 5, line 38 and in the examples thereof.

Thus, for example, polymer P-4 was prepared by polymer-analogous reaction as follows: 160 g of a 50% aqueous solution (corresponding to approximately 1 mol acid units) of polyacrylic acid (PAA, having an average molecular weight M_{w} of approximately 5000 g/mol) was placed in a round-bottom flask equipped with a mechanical stirrer (IKA stirring apparatus), thermometer, gas inlet tube, and distillation bridge. The mixture was heated to 50° C., and 210 g polyethylene glycol monomethyl ether (MPEG, having an average molecular weight M_{w} of approximately 520 g/mol) and 3 g JEFFAMINE M-2070 were added. The reaction mixture was heated to 175° C. under an N₂ stream. The water contained in the mixture as well as the reaction water were continuously distilled off under an N₂ stream. When the temperature was reached, 3 g of a 66% potassium acetate solution was added to the reaction mixture and a vacuum of 80 mbar was applied. The reaction reached completion after 2% hours. The polymer melt was allowed to solidify, or after cooling to <100° C. was combined with 1160 g water to obtain a 20% polymer solution.

Polymers P-1, P-2, and P-4 through P-8 as well as the polymers for comparative examples V-1 through V-3 were prepared in the same manner as polymer P-4.

For comparative example V-4, a commercially available plasticizer produced on a naphthalenesulfonic acid-formaldehyde condensate basis (for example, Flube OS 39, available from Bozzetto AG) was used. For comparative example V-5, a commercially available plasticizer produced on a lignosulfonate basis (for example, BORRESPERCE Ca, available from Borregaard LignoTech AG) was used. For comparative example V-6, commercially available lignosulfonates and carbohydrates (for example, BORRESPERCE Ca, available from Borregaard LignoTech AG, and molasses, available from Zuckerfabrik Frauenfeld AG) were mixed in a 55:4 ratio (dry basis) and used as plasticizer.

TABLE 2
Polymers P-1, P-2, and P-4 through P-8 used according to the invention and comparative
polymers V-1, V-2, and V-3 contain structural units A of formula (I) and structural units
B of formula (II), where R² = H, R³ = H, R⁴ = COOM, R⁶ = H, M = H⁺, Na⁺;

| | | | | | Mol-% in | |
|---|---|---|---|---|---|---|
| No. | R¹ | R⁵ | | Mw | end polymer | m/n |
| P-1 | —CH3 | —COO-PEG1000-CH3: | 99.5 | 40000 | m = 60 | 1.5 |
| | | —CO—NH-EO/PO(70/30)2000-CH3 | 0.5* | | n = 40 | |
| | | | | | p = 0 | |
| P-2 | —H | —COO-PEG1000-CH3: | 99.8 | 35000 | m = 57 | 1.3 |
| | | —CO—NH-EO/PO(70/30)2000-CH3 | 0.2* | | n = 43 | |
| | | | | | p = 0 | |
| P-4 | —H | —COO-PEG520-CH3: | 99.7 | 20000 | m = 60 | 1.5 |
| | | —CO—NH-EO/PO(70/30)2000-CH3 | 0.3* | | n = 40 | |
| | | | | | p = 0 | |
| P-5 | —H | —COO-PEG1000-CH3: | 99.7 | 30000 | m = 60 | 1.5 |
| | | —CO—NH-EO/PO(70/30)2000-CH3 | 0.3* | | n = 40 | |
| | | | | | p = 0 | |
| P-6 | —H | —COO-PEG520-CH3: | 65.7 | 25000 | m = 56 | 1.3 |
| | | —COO-PEG1000-CH3: | 34 | | n = 44 | |
| | | —CO—NH-EO/PO(70/30)2000-CH3 | 0.3 | | p = 0 | |
| P-7 | —H | —COO-PEG520-CH3: | 45.1 | 30000 | m = 58 | 1.4 |
| | | —COO-PEG1000-CH3: | 54.6 | | n = 42 | |
| | | —CO—NH-EO/PO(70/30)2000-CH3 | 0.3 | | p = 0 | |
| P-8 | —CH3 | —COO-PEG1000-CH3: | 100 | 40000 | m = 60 | 1.5 |
| | | | | | n = 40 | |
| | | | | | p = 0 | |
| V-1 | —CH3 | —COO-PEG1000-CH3: | 53.9 | 50′000 | m = 75.5 | 3.1 |
| | | —COO-PEG3000-CH3: | 45.3 | | n = 24.5 | |
| | | —CO—NH-EO/PO(70/30)2000-CH3 | 0.8 | | p = 0 | |
| V-2 | —H | —COO-PEG1000-CH3: | 29.3 | 40′000 | m = 76.3 | 3.2 |
| | | —COO-PEG3000-CH3: | 70.4 | | n = 23.7 | |
| | | —CO—NH-EO/PO(70/30)2000-CH3 | 0.3 | | p = 0 | |
| V-3 | —H | —COO-PEG1000-CH3: | 18.8 | 35′000 | m = 84.1 | 5.3 |
| | | —COO-PEG3000-CH3: | 79.9 | | n = 15.9 | |
| | | —CO—NH-EO/PO(70/30)2000-CH3 | 1.3 | | p = 0 | |

*stands for the molar ratio of various R⁵ side chains; mol-% stands for the mol-% of the individual units in the end polymer.

### 2. Mortar Tests

The effectiveness of the polymers according to the invention was tested in mortar.

| Composition of the mortar mixture (MM): | |
|---|---|
| (8 mm maximum particle size) | Quantity |
| Cement (Schweizer CEM I 42.5) | 750 g |
| Limestone filler | 141 g |
| Sand 0-1 mm | 738 g |
| Sand 1-4 mm | 1107 g |
| Sand 4-8 mm | 1154 g |

The sands, filler, and cement were mixed dry for 1 minute in a Hobart mixer. The mixing water, in which the quantity given in Table 3, relative to the cement, of a 20% aqueous solution of an additive together with polymer P-1, P-2, and P-4 through P-8 according to the invention or a comparative polymer V-1 through V-3, or the total quantity of a comparative plasticizer V-4 through V-6, was dissolved, was added over a period of 30 seconds, and mixing was continued for an additional 2.5 minutes. The 20% aqueous solution containing 20% by weight of the polymer according to the invention, or of the comparative polymer, also contained approximately 1% by weight antifoaming agent. The total wet mixing time was 3 minutes. The water/cement (w/c) value was 0.48. No plasticizers were used for comparative examples VM-7 and VM-8.

The 10% water reduction was set by adjusting a comparative mortar mixture without water-reducing additives (comparative example VM-8) to a flow table spread (0 min) of 195-200 mm by adding water. The quantity of water needed in the mortar mixture used for the tests was then reduced by 10%.

The flow table spread of the mortar was determined according to EN 1015-3. The determination of the flow table spread directly after the total wet mixing time of 3 minutes resulted in the flow table spread measured after 0 minutes.

TABLE 3
Flow table spread in mm according to EN 1015-3 after 0, 30, 60, and 90 minutes (min).

| | | | | % by weight | | | | | Δ flow table |
|---|---|---|---|---|---|---|---|---|---|
| | | | Water | based on | Flow Table Spread (mm) | | | | spead in % |
| No. | Additive | w/c | reduction | cement | 0 min | 30 min | 60 min | 90 min | after 90 min |
| M-1 | P-1 | 0.48 | 10% | 0.6 | 203 | 192 | 190 | 184 | 9.3% |
| M-2 | P-2 | 0.48 | 10% | 0.6 | 195 | 180 | 188 | 185 | 5.2% |
| M-4 | P-4 | 0.48 | 10% | 0.6 | 216 | 210 | 200 | 184 | 14.8% |
| M-5 | P-5 | 0.48 | 10% | 0.6 | 202 | 205 | 206 | 196 | 3% |
| M-6 | P-6 | 0.48 | 10% | 0.6 | 182 | 180 | 185 | 178 | 2.2% |
| M-7 | P-7 | 0.48 | 10% | 0.6 | 192 | 190 | 190 | 175 | 8.8% |
| M-8 | P-8 | 0.48 | 10% | 0.6 | 208 | 192 | 192 | 184 | 11.5% |
| VM-1 | V-1 | 0.48 | 10% | 0.6 | 230 | 216 | 190 | 172 | 25.2% |
| VM-2 | V-2 | 0.48 | 10% | 0.6 | 230 | 185 | 160 | 148 | 35.6% |
| VM-3 | V-3 | 0.48 | 10% | 0.6 | 230 | 168 | 150 | 140 | 39% |
| VM-1′ | V-1 | 0.48 | 10% | 0.35 | 205 | 175 | 162 | 146 | 28.8% |
| VM-2′ | V-2 | 0.48 | 10% | 0.34 | 198 | 164 | 152 | 148 | 25.2% |
| VM-3′ | V-3 | 0.48 | 10% | 0.34 | 200 | 165 | 155 | 145 | 27.5% |
| VM-4 | V-4 | 0.48 | 10% | 1.4 | 204 | 168 | 150 | 145 | 28.9% |
| VM-5 | V-5 | 0.48 | 10% | 2 | 198 | 150 | 145 | 138 | 30.32% |
| VM-6 | V-6 | 0.48 | 10% | 1.8 | 202 | 152 | 145 | 140 | 30.7% |
| VM-7 | — | 0.48 | 10% | — | 160 | 146 | 145 | 132 | 17.5% |
| VM-8 | — | 0.54 | — | — | 204 | 190 | 183 | 177 | 13.2% |

The % by weight of the additives is stated as % by weight of a 20% aqueous solution containing polymers P-1, P-2, and P-4 through P-8 and V-1 through V-3, or as the % by weight of the total weight of additives V-4 through V-6; in each case the % by weight is relative to the cement.

The results in Table 3 show that the polymers according to the invention have excellent plasticizing properties compared to conventional polymers V-1, V-2, and V-3 and comparative plasticizers V-4 through V-6. This is primarily demonstrated by the values of the flow table spread after 30 to 90 minutes, in which the flow table spread for polymers P-1, P-2, and P-4 through P-8 remains relatively constant over 90 minutes, and decreases by a maximum of 15% compared to the initial flow table spread after 0 minutes. Particularly good results were obtained with polymers P-1, P-2, and P-6 through P-7. That is, particularly good results are obtained when the polymer has a low m/n ratio of 1.2 to 1.6.

When comparative polymers V-1 through V-3 are dosed in the same quantities as for the polymers according to the invention (Example Nos. VM-1 through VM-3), the initial flow table spread is too high, and the mortar mixture may separate and is therefore unusable. In addition, up to a period of 90 minutes the flow table spread decreases by more than 25% compared to the initial flow table spread. This decrease in flow table spread is not desired in practice.

When comparative polymers V-1 through V-3 are dosed at lower quantities than for the polymers according to the invention (Example Nos. VM-1′ through VM-3′), so that at the start the initial flow table spread is equal to that of the polymers according to the invention, up to a period of 90 minutes the flow table spread likewise decreases by more than 25% compared to the initial flow table spread, which is not desired in practice.

For comparative examples VM-4 through VM-6 based on lignin sulfonates or naphthalene sulfonates, using a corresponding dosage the initial flow table spread was set to approximately 200 mm to allow comparison with the examples of the invention. To achieve this initial flow table spread, the conventional plasticizers must be dosed in much higher quantities. In addition, over a period of 90 minutes the flow table spread decreased by more than 28% compared to the initial flow table spread.

No water-reducing additives or polymer P were used for comparative example VM-7. Thus, at the same w/c value as for the examples of the invention, the initial flow table spread is much lower, and the mixture is hardly workable.

Likewise, no water-reducing additives or polymer P were used for comparative example VM-8, but an initial flow table spread of approximately 200 mm was achieved by adding more water (w/c value of 0.54, and thus 10% higher than for the preceding examples). The workability is therefore approximately in the desired range, although according to experience, mortar and concrete compositions having a higher w/c value, and thus a higher water fraction, have much lower strength values and therefore poorer mechanical properties.

Thus, it is desirable to produce concrete and mortar mixtures which have approximately the same workability as achieved for VM-8, which, however, require less water, i.e., in which the water reduction is approximately 5 to 15%, and which therefore have better strength values. This is achieved for polymers P-1, P-2 and P-4 through P-8 according to the invention.

### 3. Concrete Tests

The effectiveness of the polymers according to the invention was also tested in concrete.

| Composition of the concrete mixture (CM): | |
|---|---|
| (32 mm maximum particle size) | Quantity |
| Cement (Schweizer CEM I 42.5) | 750 g |
| Limestone filler | 235 g |
| Sand 0-1 mm | 1170 g |
| Sand 1-4 mm | 1355 g |
| Sand 4-8 mm | 515 g |
| Gravel 8-16 mm | 608 g |
| Gravel 16-32 mm | 795 g |

The gravels, sands, filler, and cement were mixed dry for 30 seconds in a tumbler mixer. The mixing water, in which the quantity given in Table 4, relative to the cement, of a 20% aqueous solution of an additive together with polymer P-1 according to the invention or comparative polymer V-1, or the total quantity of a formulation F-P1 (corresponding to a 28.25% aqueous solution) or a comparative plasticizer V-5, was dissolved, was added over a period of 30 seconds, and mixing was continued for an additional 1.5 minutes. The 20% aqueous solution containing 20% by weight of the polymer according to the invention, or of the comparative polymer, also contained approximately 1% by weight antifoaming agent. The total wet mixing time was 2 minutes. The water/cement (w/c) value was 0.6. The 10% water reduction was set by adjusting a concrete mixture without water-reducing additives to a flow table spread (0 min) of 500-600 mm by adding water. The quantity of water needed was then reduced by 10%.

Formulation F-P1 contains 13% by weight polymer P-1, 13% by weight of a plasticizer produced on a lignosulfonate basis (BORRESPERCE Ca, available from Borregaard LignoTech AG), and 2.25% by weight molasses (available from Zuckerfabrik Frauenfeld AG) in 71.75% by weight water, which corresponds to a 28.25% aqueous solution.

Since the composition of the concrete mixture contains gravel with a maximum particle size of 32 mm, the flow table spread of the concrete was determined according to EN 12350-5. The determination of the flow table spread directly after the wet total mixing time of 2 minutes resulted in the flow table spread measured after 0 minutes.

TABLE 4
Flow table spread in mm according to EN 12350-5 after 0, 30, 60, and 90 minutes (min)

| | | | | % by weight | | | | | Δ flow table |
|---|---|---|---|---|---|---|---|---|---|
| | | | Water | based on | Flow Table Spread (mm) | | | | spead in % |
| No. | Additive | w/c | reduction | cement | 0 min | 30 min | 60 min | 90 min | after 90 min |
| B-1 | P-1 | 0.6 | 10% | 0.6 | 540 | 510 | 490 | 460 | 14.8% |
| B-2 | F-P1 | 0.6 | 10% | 0.6 | 520 | 460 | 430 | 430 | 17.3% |
| VB-1 | V-1 | 0.6 | 10% | 0.35 | 510 | 440 | 410 | 380 | 25.5% |
| VB-5 | V-5 | 0.6 | 10% | 1.4 | 520 | 410 | 390 | 360 | 30.7% |
| VB-8 | — | 0.67 | | — | 530 | 460 | 440 | 420 | 20.7% |

The results in Table 4 show that longer workability is also achieved in concrete compositions containing polymer P-1 than for compositions containing a conventional plasticizer and no polymer P. Longer workability is achieved even with formulations containing polymer P-1 (F-P1).

For comparative example VB-1, a lower quantity of polymer must be dosed in order to achieve the desired initial flow table spread. However, in that case the workability greatly decreases over time, and the concrete composition has poor workability.

For comparative example VB-5 the dosage is much higher in order to achieve the desired initial flow table spread. However, the workability decreases greatly over time, and the concrete composition is likewise hardly workable.

For comparative example VB-8 it is necessary to use a 10% higher quantity of water to achieve the desired initial flow table spread. On the one hand, the decrease in workability is greater than for polymer P according to the invention, and on the other hand the strength of the produced concrete composition decreases greatly due to the increased water demand, and the concrete composition therefore has poorer mechanical properties (Table 5).

For determining the mechanical properties, the pressure-tightness of cubes (120×120×120 mm) was determined using a hydraulic press (Table 5).

TABLE 5
Pressure-tightness in N/mm² after 1 and 28 days (d) (concrete
temperature 30° C.)

| | |
|---|---|
| | Pressure- |
| % by weight | tightness |
| based on | (N/mm²) |

| No. | Additive | w/c | cement | 1 d | 28 d |
|---|---|---|---|---|---|
| B-2 | F-P1 | 0.6 | 0.6 | 19.8 | 38.9 |
| VB-8 | — | 0.67 | — | 15.1 | 31.7 |

It is apparent from Table 5 that a concrete mixture which does not contain water-reducing additives but which has a fairly high w/c value in order to obtain the desired initial flow table spread has much poorer strength values after 1 day and after 28 days, compared to a concrete mixture containing formulation F-P1 which includes polymer P-1.

Of course, the invention is not limited to the exemplary embodiments illustrated and described. It is understood that the above-referenced features of the invention may be used not only in the particular stated combination, but also in other modifications, combinations, and revisions, or alone, without departing from the scope of the invention.

1. A polymer P for prolonging the flowability of water-reduced aqueous hydraulically setting compositions, the polymer P comprising:
a) m mol-% of at least one acid unit A of formula (I); 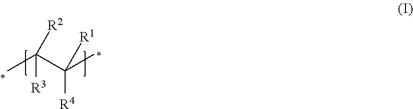
and
b) n mol-% of at least one structural unit 13 of formula (II); 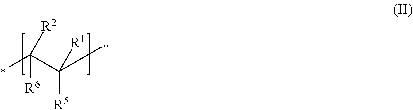
and optionally
c) p mol-% of at least one structural unit C;
wherein R¹ and R² in formula (I) or formula (II) independently is H, COOM, CH₂COOM, or an alkyl group containing 1 to 5 carbon atoms,
wherein M is H, an alkali metal, an alkaline earth metal, ammonium, alkylammonium, or mixtures thereof:
wherein R³ in formula (I) independently is H, CH₃, COOM, or CH₂COOM; and
wherein R⁴ in formula (I) independently is a radical of carboxylic acid,
or wherein R³ together with R⁴ forms a ring to give —CO—O—CO—;
wherein R⁵ in formula (II) independently is a radical of formula (III)
—(CH₂)ₓ—R⁷—(R⁸O)_{y}—R⁹ (III)
wherein R⁷ independently is an ester, ether, amide, or imide connecting element;
wherein R⁸ is a C₂-C₆ alkylene group or a mixture thereof,
wherein R⁹ is H, a C₁-C₁₂ alkyl or cycloalkyl radical, a C₇-C₂₀ alkylaryl or aralkyl radical, a substituted or unsubstituted aryl radical, or a monofunctional organic radical containing 1 to 30 C atoms and optionally containing heteroatoms;
wherein x independently is 0 or 1;
wherein y independently is 3-250;
wherein R⁶ in formula (II) is H, CH₃, COOM, CH₂COOM, or a substituent such as defined above for R⁵;
wherein m, n, and p independently stand for numbers,
wherein a sum m+n+p=100, and in >0, n>0, and p≧0; and
wherein a ratio m/n is between 1.2 and 1.7.

2. The polymer of claim 1, wherein the polymer P is obtainable by esterification and/or amidation of a polycarboxylic acid or an analog thereof, using a polymer-analogous reaction.

3. The polymer of claim 2, wherein the polymer P is obtainable by reacting
(a) at least one polycarboxylic acid or an analog of a polycarboxylic acid; and
(b) at least one compound selected from the group consisting of a monohydroxy compound E of formula (IV)
HO—(R⁸O)_{y}—R⁹ (IV),
and a monoamine compound F of formula (V);
NH₂—(R⁸O)_{y}—R⁹ (V),
and optionally,
(c) at least one further compound D,
wherein R⁸ in formula (III) or formula (IV) independently is a C₂-C₄ alkylene group having any possible sequence of the (R⁸O) units;
wherein R^{9′} in formula (IV) is a C₁-C₁₂ alkyl or cycloalkyl radical, a C₇-C₂₀ alkylaryl or aralkyl radical, a substituted or unsubstituted aryl radical, or a monofunctional organic radical containing 1 to 30 C atoms and optionally containing heteroatoms;
wherein R⁹ in formula (V) is H, a C₁-C₁₂ alkyl or cycloalkyl radical, a C₇-C₂₀ alkylaryl or aralkyl radical, or a substituted or unsubstituted aryl radical, or a monofunctional organic radical containing 1 to 30 C atoms and optionally containing heteroatoms; and
wherein y independently is a value of from 3 to 250.

4. The polymer of claim 3, wherein the analog of polycarboxylic acid for polymer P is selected from the group consisting of acid salts, acid halides, and acid anhydrides.

5. The polymer of claim 1, wherein the polymer P is obtainable by a radical polymerization reaction.

6. The polymer of claim 1, wherein in polymer P, R¹ is H or CH₃, R², R³, and R⁶ is H, and R⁴ is COOM.

7. The polymer of claim 1, wherein R⁵ contains at least 30 mol-% C₂H₄O units, relative to the total molar quantity of all (R⁸O) units.

8. The polymer of claim 1, wherein the polymer P contains 30 to 66 mol % of acid unit A, 20 to 50 mol % of structural unit B of formula (II), and optionally 0 to 40 mol-% of structural unit C, in each case relative to a total molar quantity of structural units A, B, and C in the polymer P.

9. (canceled)

10. (canceled)

11. A method for prolonging the flowability of water-reduced aqueous hydraulically setting compositions, the method comprising:
adding the polymer P of claim 1 to an aqueous hydraulically setting composition Z1 containing water and a, hydraulic binder,
wherein the aqueous hydraulically setting composition Z1 is identical to an aqueous hydraulically setting reference composition Z2 except that the aqueous hydraulically setting composition Z1 has a water content that is 5-15% less than a water content of the aqueous hydraulically setting reference composition Z2,
wherein Z1 and Z2:
i.) each have a flow table spread directly after admixture of water of 180 to 220 mm according to EN 1015-3, or 450 to 550 mm according to EN 12350-5, and
ii.) each have a at least a maximum difference in the flow table spread directly after admixture of the water compared to a flow table spread after 90 minutes of 15% according to EN 1015-3, or 20% according to EN 12350-5.

12. An aqueous hydraulically setting composition Z1 comprising: the polymer P of claim 1, water and a hydraulic binder,
wherein the aqueous hydraulically setting composition Z1 is identical to an aqueous hydraulically setting reference composition Z2 except that the aqueous hydraulically setting composition Z1 contains the polymer P, and contains 5-15% less water than the aqueous hydraulically setting reference composition Z2, and
wherein Z1 and Z2:
i.) each have a flow table spread directly after admixture of the water of 180 to 220 mm according to EN 1015-3 or 450 to 550 mm according to EN 12350-5, and
ii.) each have at least a maximum difference in the flow table spread directly after admixture of the water compared to a flow table spread after 90 minutes of 15% according to EN 1015-3, or 20% according to EN 12350-5.

13. The aqueous hydraulically setting composition Z1 of claim 12, wherein the hydraulic binder is selected from the group consisting of cement; mixtures of cement with fly ash, fumed silica, slag, granulated blast furnace slag, or limestone filler; gypsum; and quicklime.

14. The aqueous hydraulically setting composition Z1 of claim 12, further comprising at least one plasticizing agent selected from the group consisting of lignosulfonate, naphthalenesulfonic acid-formaldehyde condensate, sulfonated melamine-formaldehyde condensate, molasses, and gluconate.
